(19) United States
(12) Patent Application Publication
(10) Pub. No.: US 2011/0178556 A1
(43) Pub. Date: Jul. 21, 2011

(54) FIXATION SYSTEM

(76) Inventor: Henrik Hansson, Vreta Kloster (SE)

(21) Appl. No.: 13/000,431

(22) PCT Filed: Jun. 2, 2009
(86) PCT No.: PCT/SE2009/050646

§ 371 Date: Feb. 15, 2011

(30) Foreign Application Priority Data
Jul. 1, 2008 (SE) 0801558-8

(51) Int. Cl. A61B 17/80

(52) U.S. Cl. 606/286

(57) ABSTRACT

The present invention relates to a system for internal fixation of bone fragments (4a, 4b) in a fracture (3) or of bones in a bone structure, which system comprises a plate (1) and at least two fixing elements (2) which can be screwed into the plate. With the object of providing a fixation system which affords largely unlimited freedom of choice as to where fixing elements may be screwed into the plate, the plate (1) is adapted to being applied abutting against bone fragments (4a, 4b) in the fracture (3) or against bones in the bone structure and is made of a material which can be shaped to the bone fragments or bones against which the plate is intended to abut. The material of the plate (1) is UHMWPE (ultra high molecular weight polyethylene) with an elasticity which makes it possible to screw fixing elements in the form of screws (2) in at any desired locations in the plate in such a way that the screws are locked in the plate by friction with respect to movements in axial, rotary and angular directions. Screwing the screws (2) into bone fragments (4a, 4b) in the fracture (3) or into bones in the bone structure results in fixation of said bone fragments or bones.

### BACKGROUND TO THE INVENTION

The present invention relates to a system for internal fixation of bone fragments in a fracture or of bones in a bone structure, which system comprises a plate and at least two fixing elements which can be screwed into the plate.

Plates for such fixation systems are most commonly made of metal but may also be made of plastic. The plates may be used externally and internally. Plates made of plastic for internal use are provided with predrilled holes for fixing elements. The disadvantage of predrilled holes is that the locations in the plate where fixing elements can be screwed in are predetermined and the possibility of successful use depends largely on the plate being of optimum size and shape in relation to the anatomy of the respective patient.

### BRIEF SUMMARY OF THE INVENTION

The object of the present invention is to propose a fixation system as above which affords largely unlimited freedom of choice of locations in the plate where fixing elements can be screwed in.

To this end, according to the invention, the plate is adapted to being applied abutting against bone fragments in the fracture or against bones in the bone structure and is made of a material which can be shaped to the bone fragments or bones against which the plate is intended to abut. The material of the plate is UHMWPE (ultra high molecular weight polyethylene) with an elasticity which allows fixing elements in the form of screws to be screwed in at any desired location in the plate in such a way that the screws are locked in the plate by friction with respect to movements in axial, rotary and angular directions. Screwing the screws into bone fragments in the fracture or into bones in the bone structure results in fixation of said bone fragments or bones.

The fact that the plate has no predrilled holes for the screws thus also results in effective locking of the screws when they are screwed into the plate, irrespective of where in the plate they are screwed in. With the object of further improving their locking, the screws according to a further version of the invention are self-drilling and self-tapping and can therefore themselves engage holes in the plate for their effective locking. No holes need to be predrilled.

With the object likewise of further improving said fixation of bone fragments or bones, the screws according to a further version are of compression type, which enables them to draw bone fragments and/or bone fragments and the plate towards one another or to draw bones and the plate towards one another.

Other objects and advantages of the invention will be apparent to one skilled in the art who examines the attached drawings and the following detailed description of preferred embodiments.

### BRIEF DESCRIPTION OF THE DRAWINGS

FIGS. 1 and 2 are schematic views in perspective of a conceivable version of a plate forming part of a fixation system according to the present invention for a radius fracture.

FIG. 3 is a schematic view in perspective of the version depicted in FIGS. 1 and 2 of a plate forming part of a fixation system according to the present invention for a radius fracture after the plate has been caused to abut against, and transversely across a fracture in, a radius.

FIG. 4 is a schematic view of a possible version of a fixing element which forms part of the fixation system according to the present invention and takes the form of a screw shown slid onto a guide rod.

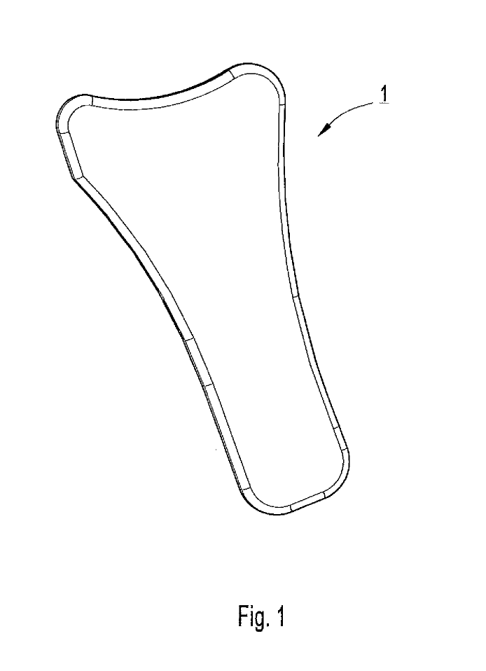

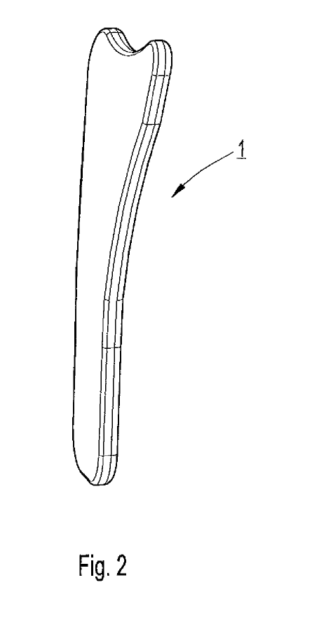

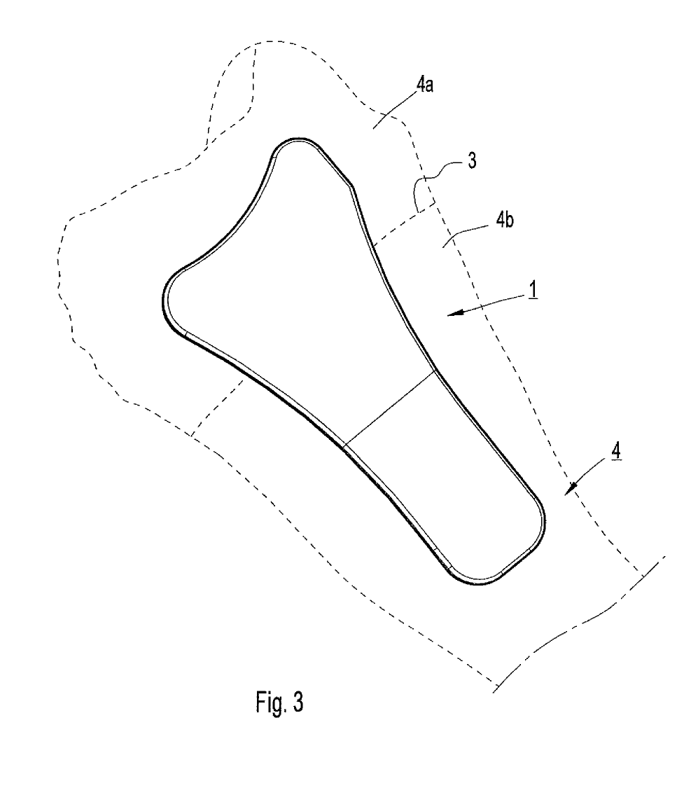

### DETAILED DESCRIPTION OF PREFERRED EMBODIMENTS OF THE INVENTION

The present invention relates to a fixation system for internal fixation of bone fragments in a fracture or of bones in a bone structure. The fixation system comprises a plate 1 and at least two fixing elements 2 which can be screwed into the plate. The fractures to which the fixation system according to the invention is applicable are of any kind where a plate 1 and fixing elements 2 may be relevant. Examples of fractures which may here be relevant are collarbone fractures, fractures in the humerus, fractures in elbow joints, fractures in forearms, e.g. radius fractures, wrist fractures or fractures in the fingers. Correspondingly, the bone structures to which the fixation system according to the invention may be applicable are of any kind where a plate 1 and fixing elements 2 may be relevant, e.g. bone structures in wrists or hands where so-called fusion plates (e.g. three-corner and four-corner fusion plates) of various kinds are used.

The plate 1 of the fixation system according to the present invention is adapted to abutting against bone fragments in the fracture or against bones in the bone structure concerned, i.e. it is intended, as previously mentioned, for internal fixation. The plate 1 is also preferably of appropriate shape and size for the intended application. In the version depicted for a radius fracture, the plate is therefore, inter alia, elongate and somewhat wider at one end. The plate 1 is made of a material which can be shaped to the bone fragments or bones against which the plate 1 is intended to abut, i.e. it can be shaped to still better conform to the anatomy of said bones. In addition, the elasticity of the material of the plate 1 is such as to allow fixing elements 2 to be screwed in at any desired locations in the plate 1 in such a way that the fixing elements are locked in the plate 1 by friction with respect to movements in axial, rotary and angular directions. The material of the plate 1 has at the same time to be of sufficient flexural rigidity to allow, for example, a fracture to heal. The material for the plate 1 is polyethylene, i.e. UHMWPE (ultra high molecular weight polyethylene), which is approved for medical use both externally and internally, i.e. it is biocompatible, and which is also transparent to radiography. UHMWPE is also a tough and solid material with no pores. UHMWPE thus meets the mechanical requirements as regards tensile and/or compressive strength, rigidity, fatigue resistance, wear resistance and dimensional stability which are applicable to materials to be used inter alia for fixation systems of the kind here concerned, i.e. it normally has, for example, a rupture limit of at least 35, an elasticity modulus of 4-12 and an elongation of at least 300 percent.

For effective locking of fixing elements 2 in the plate 1 which is composed of formable material with the elasticity indicated, the plate is with advantage made without predrilled holes for fixing elements 2. Instead, holes in the plate 1 for fixing elements 2 are created by the fixing elements at the time when the fixation system according to the invention is being surgically inserted in a patient.

For the purpose indicated above, the fixing elements of the fixation system according to the present invention take the form of screws 2. The screws 2 take the form with advantage of self-drilling and self-tapping screws 2. The fixing elements or screws 2 are also of compression type adaptable, as indicated above and depending on the particular application, to drawing bone fragments and/or bone fragments and the plate towards one another or to drawing bones and the plate towards one another. Screwing the fixing elements 2 in at any desired locations in the plate and into bone fragments in the fracture or into bones in the bone structure thus results in locking of the fixing elements in the plate and fixation of said bone fragments or bones.

In cases where the plate 1 of the fixation system according to the present invention is to be used as a distal radius plate, it may possibly be provided with at least one predrilled distal or proximal hole (not depicted), but no predrilled proximal or distal holes for fixing elements 2. Where it is used as a fusion plate, there will, as previously indicated, be no predrilled holes. The result is optimum possibilities for choice of locations for fixing elements 2.

The plate 1 of the fixation system according to the present invention may also be provided with at least two fixing portions (not depicted) for crosswise application of at least two fixing elements 2. To this end, the fixing portions may be somewhat angled relative to the rest of the plate 1, or the fixing elements 2 may be screwed in through the fixing portions at an angle relative to the rest of the plate 1. The fixing portions may comprise small portions, lugs etc. which protrude sideways from the plate 1.

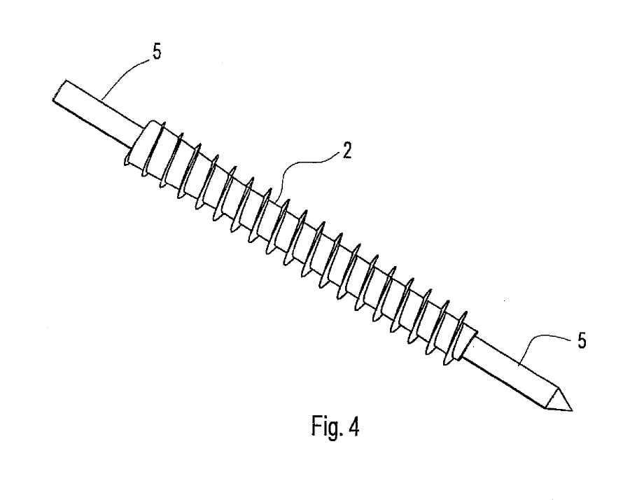

The configuration of the fixing elements or screws 2 such that they are self-drilling and self-tapping is known per se and is therefore not described in more detail here. In the version depicted, the screws 2 are fully threaded, making it easier to cope with cyclic loading which may occur during healing. In the version depicted, to achieve the compression effect indicated above, the thread pitch varies in the longitudinal direction of the screws 2. This thread pitch varies in such a way that it increases towards the tip of the screws 2. The screw threads nearest to the screw tip thus penetrate bone tissue more quickly than the finer screw threads further away from the screw tip. Compression takes place progressively as the screw is screwed into bone tissue. Varying thread pitch may also be applicable even if the screws are not fully threaded. Alternative solutions for achieving compression are also within the scope of the present invention. The screws 2 may be cannulated or uncannulated, and in the latter case they may be solid and have no duct running through them in their longitudinal direction for a guide rod 5. The screw 2 depicted in FIG. 4 has such a duct for a guide rod 5. The screw 2 depicted in FIG. 4 also has no screwhead, in order to minimise tissue damage, but it may also be provided with a screwhead where necessary or desired, in which case the screwhead may also be threaded.

Briefly, the fixation of a bone fracture, e.g. a fracture 3 in a radius 4, by means of the fixation system described above, may be effected as follows:

One or two incisions are made which run across parts of bone fragments 4a and 4b on the respective sides of the fracture 3.

The plate 1 is applied and a sharp-pointed fine guide rod 5 which locks is drilled through the plate at any desired location thereon and into each of the bone fragments 4a, 4b, i.e. at at least two points in the plate, in order to place and hold the plate in correct position relative to the fracture 3 and, as previously indicated, to lock the bone fragments. Alternatively, the plate 1 may be applied and held in correct position by means of a guide rod 5 in a predrilled distal or proximal hole and a guide rod 5 placed at any desired location. The guide rods 5 may be fully threaded or not and may have a drill bit or trocar tip. A cannulated screw 2 is slid onto the respective guide rod 5 and is screwed into and through the plate 1 and into the respective bone fragment 4a or 4b. The screws 2 are screwed in in such a way as not to create any forces which might risk shifting the position of the plate 1. Effective locking of the screws in the plate is achieved by the fact that the screws 2 are with advantage self-drilling and self-tapping and that the material of the plate 1 has the elasticity indicated above. The further fact that the screws 2 are with advantage of compression type as above achieves also a drawing together of the bone fragments 4a, 4b and the plate 1 towards one another and a fixation of the bone fragments. The screws 2 are applied in such a way that they run through the two bone fragments 4a, 4b, thereby naturally also drawing the bone fragments towards one another and fixing them. The guide rod 5 can then be drawn out or screwed out. Further guide rods 5 for further cannulated screws 2 may be used at any desired locations on the plate 1.

The above method with guide rods 5 and cannulated screws 2 is used with advantage in cases where the fracture is in very hard bone. Alternatively, uncannulated solid screws may be screwed in through the plate 1 at any desired locations thereon and into the bone fragments 4a, 4b either immediately or after drilling and reading off by means of a gauge rod, for locking in the plate and fixation of the bone fragments.

The operation is completed by suturing and bandaging the surgical wound.

It will be obvious to one skilled in the art that the fixation system according to the present invention may vary within the scope of the claims set out below without departing from the idea and objects of the invention. Thus, as indicated above, the shape and size of the constituent parts of the fixation system may vary depending on how the device is to be used, i.e. the type of fracture concerned, whether the patient is a child or an adult, the patient's anatomy, how many fixing elements are to be or need to be used and at what angles relative to the fracture, etc.

1. A system for internal fixation of bone fragments (4a, 4b) in a fracture (3) or of bones in a bone structure,
which system comprises a plate (1) and at least two fixing elements (2),
which plate (1) is adapted to being applied abutting against bone fragments (4a, 4b) in the fracture (3) or against bones in the bone structure and is made of a material which can be shaped to the bone fragments or bones against which the plate is intended to abut,
which fixing elements take the form of screws (2) for screwing into the plate (1) and into bone fragments (4a, 4b) in the fracture or into bones in the bone structure,
wherein the plate (1) is made of UHMWPE (ultra high molecular weight polyethylene) and has an elasticity which makes it possible to screw the screws (2) in at any desired locations in the plate in such a way that the screws are locked in the plate by friction with respect to movements in axial, rotary and angular directions and in such a way that screwing the screws into bone fragments (4a, 4b) in the fracture or into bones in the bone structure results in fixation of said bone fragments or bones.

2. A system according to claim 1, wherein the plate (1) is a so-called distal radius plate with at least one predrilled distal or proximal hole but no predrilled proximal or distal holes.

3. A system according to claim 1, wherein the plate (1) is a so-called fusion plate with no predrilled holes.

4. A system according to claim 1, wherein the plate (1) is provided with at least two fixing portions for crosswise application of at least two screws (2).

5. A system according to claim 1, wherein the screws taking take the form of self-drilling, self-tapping screws (2).

6. A system according to claim 1, wherein the screws (2) are of compression type.

7. A system according to claim 1, wherein each screw (2) is fully threaded with varying thread pitch in the longitudinal direction.

8. A system according to claim 7, wherein the thread pitch of each screw (2) increases towards the tip of the screw.

9. A system according to claim 1, wherein each screw (2) is cannulated.

10. A system according to claim 1, wherein each screw (2) is solid.

11. A system according to claim 1, wherein each screw (2) has no screwhead.
